(19) United States
(12) Patent Application Publication
(10) Pub. No.: US 2026/0085054 A1
(43) Pub. Date: Mar. 26, 2026

(54) DERIVATIVE OF HISPIDIN FROM SANGHUANGPORUS SANGHUANG, AND PREPARATION METHOD AND USE THEREOF

(71) Applicant: China Three Gorges University, Yichang (CN)

(72) Inventors: Chengfu Yuan, Yichang (CN); Zhiwen Zhou, Yichang (CN); Yuting Liu, Yichang (CN); Hongqi Zhang, Yichang (CN)

(21) Appl. No.: 19/401,409

(22) Filed: Nov. 26, 2025

(30) Foreign Application Priority Data
Oct. 31, 2025 (CN) 202511583203.2

(51) Int. Cl. C07D 309/36; A61K 31/366; A61P 3/06; C07D 405/06

(52) U.S. Cl. CPC C07D 309/36; A61K 31/366; A61P 3/06; C07D 405/06

(57) ABSTRACT

The present disclosure relates to the technical field of drug synthesis, and particularly relates to a Hispidin derivative, and a preparation method and use thereof. The Hispidin derivative has a structure of formula (I):

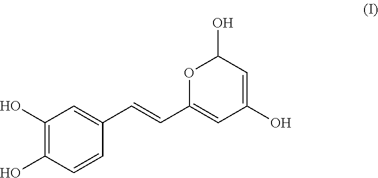 [SMILES: OC1=CC(O)OC(/C=C/c2ccc(O)c(O)c2)=C1]

The present disclosure employs 4-hydroxy-6-methyl-2-pyrone and 3,4-dihydroxybenzaldehyde as starting raw materials to synthesize a series of compounds with styrylpyrone as a backbone. The preparation method provided by the present disclosure has simple steps, is easy to operate, has good feasibility, has the potential for large-scale industrial application, and has a broad market prospect. The present disclosure further provides use of the preparation method described above and the resulting Hispidin derivative in preparation of a lipid-lowering drug. The aryl compound provided by the present disclosure has a good lipid-lowering therapeutic effect and can be used for preparing a lipid-lowering drug, thus having good social benefits.

### TECHNICAL FIELD

The present disclosure relates to the technical field of drug synthesis, and particularly relates to a Hispidin derivative thereof, and a preparation method and use thereof.

### BACKGROUND

Under an obesity status, the abnormal accumulation of adipose tissues in the body leads to the imbalance of lipid metabolism. Commonly used lipid-reducing drugs in clinic includes Statin, but such drugs have side effects such as gastrointestinal reactions and hepatotoxicity. Traditional Chinese herbal medicines contain a variety of natural chemical ingredients that have lipid-lowering efficacies. The natural ingredients have greater safety, providing a natural option for seeking safe and effective lipid-lowering compounds.

Hispidin, a styrylpyrone compound from Sanghuangporus sanghuang, is a protein kinase C (PKC) inhibitor that can reverse palmitate-induced glucose uptake inhibition and inhibit intracellular lipid accumulation, thereby achieving a lipid-lowering effect. Hispidin contains a lactone ring structure such as pyrone, which is similar to a six-membered lactone ring structure that is an essential group in a Statin lipid-lowering drug, and both them have lipid-lowering effects. The Sanghuangporus sanghuang has a relatively long growth cycle and the natural resource thereof is limited. Furthermore, the Hispidin extracted from the Sanghuangporus sanghuang is low in content and purity, so there is certain difficulty in obtaining Hispidin with stable quality therefrom. In order to obtain high-purity and high-yield Hispidin and solve its source problem, the present disclosure synthesizes a Hispidin derivative by an organic chemical reaction of 4-hydroxy-6-methyl-2-pyrone and 3,4-dihydroxybenzaldehyde to obtain a lead compound with a good lipid-lowering effect, which improves its clinical efficiency of lipid-lowering effect, and provides theoretical support for the search for new lipid-lowering drugs.

### SUMMARY

The present disclosure provides a Hispidin derivative and a preparation method and use thereof, which solves the problem of low content and low purity of Hispidin extracted from Sanghuangporus sanghuang. The Hispidin derivative provided by the present disclosure has a certain lipid-lowering effect.

The present disclosure has the following beneficial effects:

1. The present disclosure uses the Hispidin as a lead compound to design and synthesize a series of styrylpyrone derivatives, which exhibit good lipid-lowering activity.

2. The Hispidin derivative is prepared by a chemical synthesis route, overcoming the technical bottlenecks of long growth cycle and limited natural resource of the Sanghuangporus sanghuang, and low extraction purity and unstable yield of the Hispidin. A method for preparing a high-purity, high-yield, and batch-stable compound is achieved.

3. The synthesized styrylpyrone compound has good drug compatibility and stability, and can be used for preparing a novel lipid-lowering drug.

### BRIEF DESCRIPTION OF THE DRAWINGS

FIG. 1 is a hydrogen spectrogram of a compound D3 obtained in Example 3.

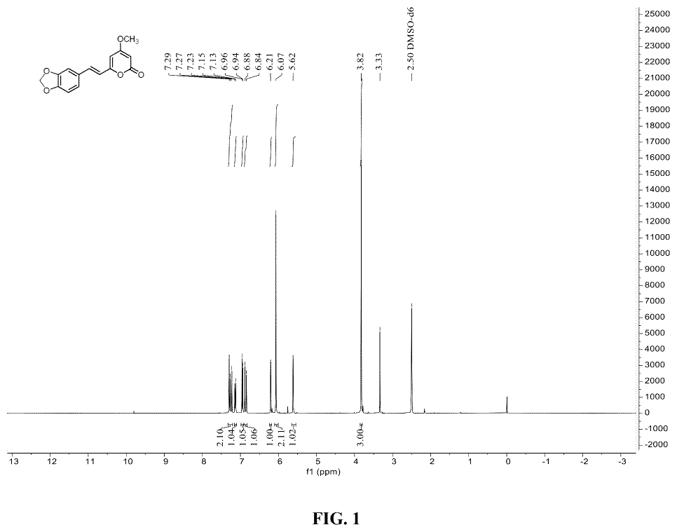

FIG. 2 is a hydrogen spectrogram of a compound D4 obtained in Example 4.

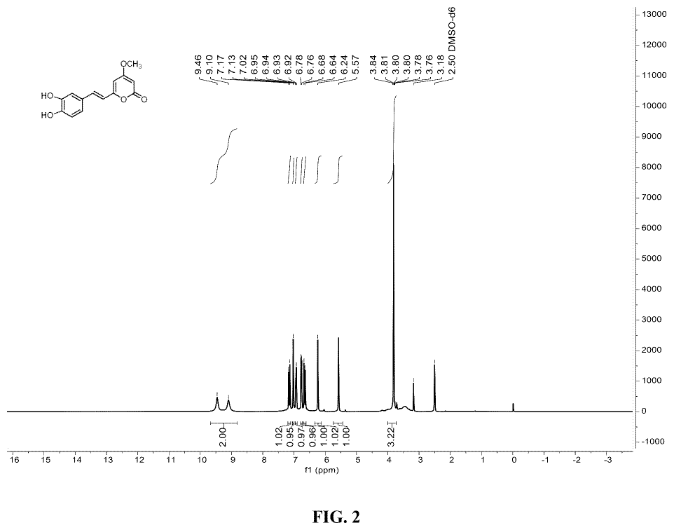

FIG. 3 is a hydrogen spectrogram of a compound D5 obtained in Example 5.

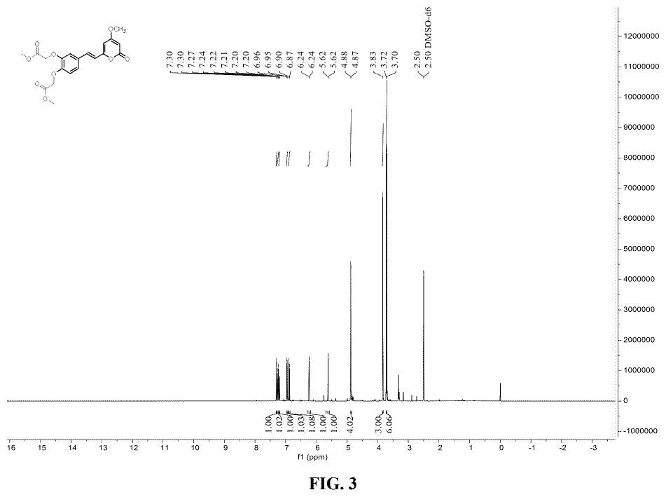

FIG. 4 is a hydrogen spectrogram of a compound D6 obtained in Example 5.

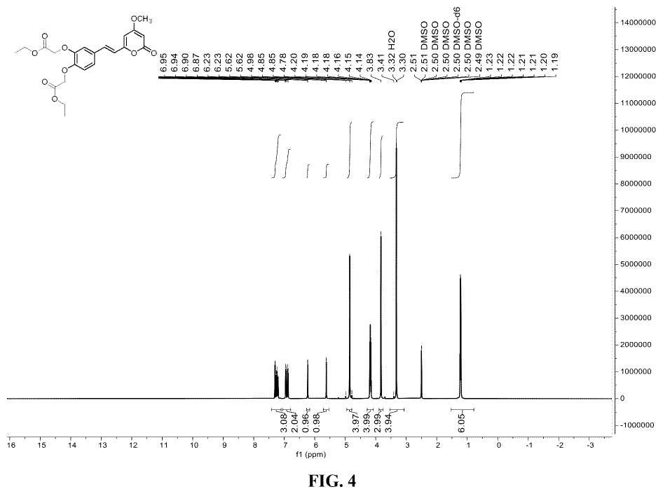

FIG. 5 is a hydrogen spectrogram of a compound D7 obtained in Example 5.

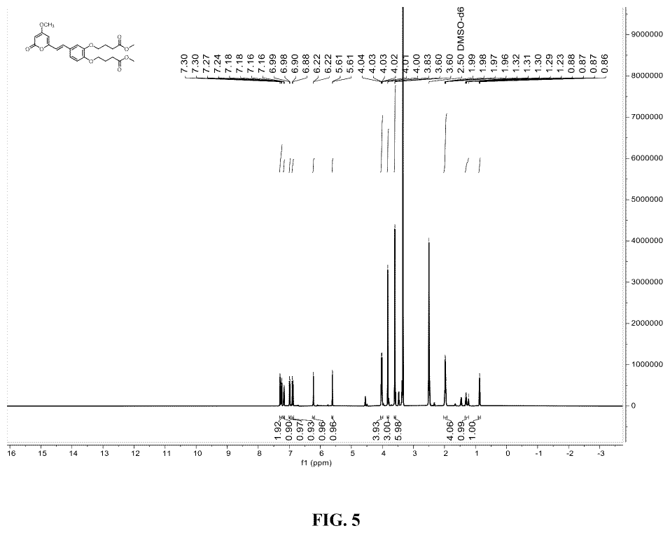

FIG. 6 is a hydrogen spectrogram of a compound D8 obtained in Example 5.

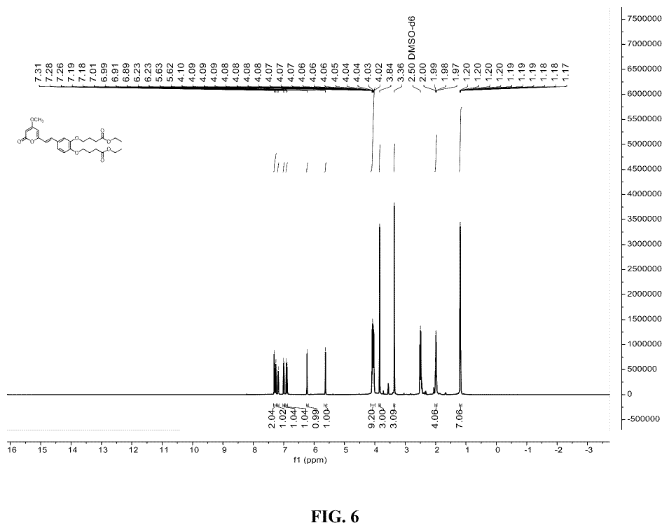

FIG. 7 is a hydrogen spectrogram of a compound D9 obtained in Example 5.

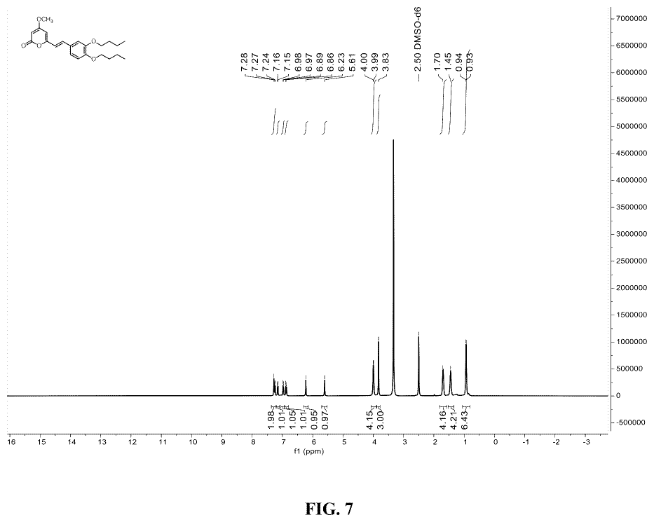

FIG. 8 is a hydrogen spectrogram of a compound D10 obtained in Example 5.

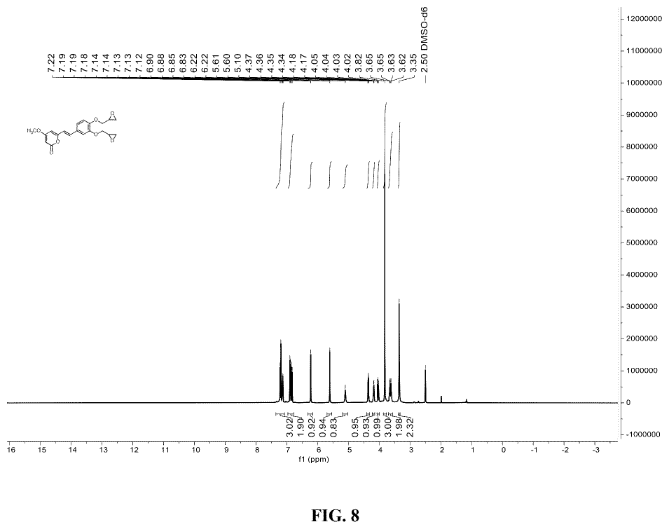

FIG. 9 is a hydrogen spectrogram of a compound D11 obtained in Example 5.

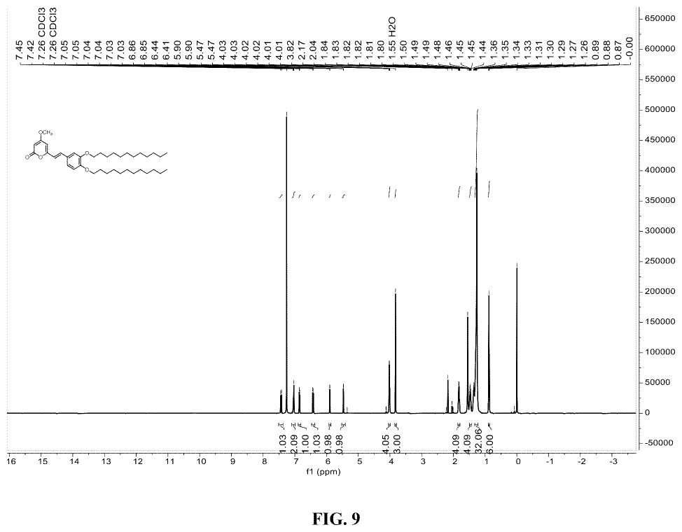

FIG. 10 is a hydrogen spectrogram of a compound D12 obtained in Example 5.

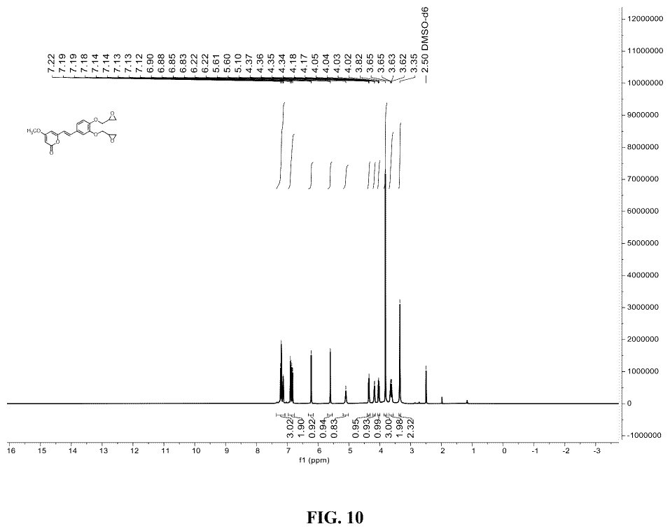

FIG. 11 is a hydrogen spectrogram of a compound D13 obtained in Example 5.

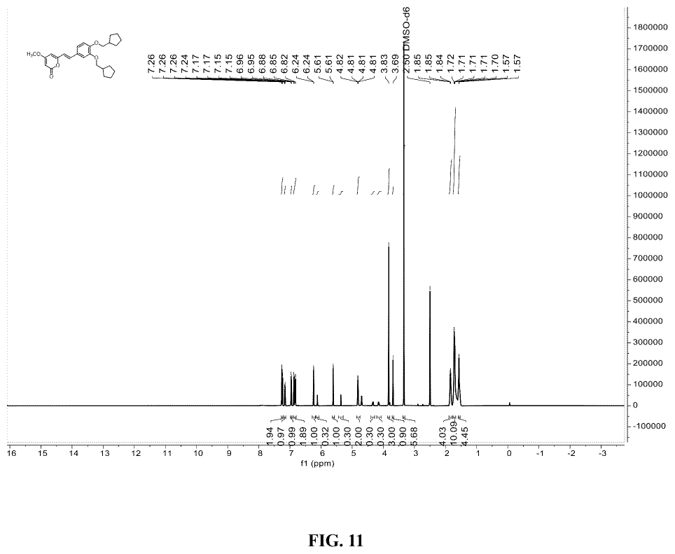

FIG. 12 is a hydrogen spectrogram of a compound D14 obtained in Example 5.

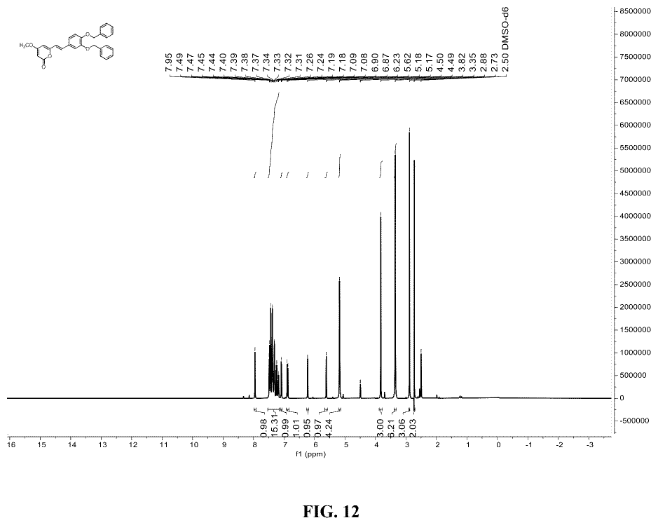

FIG. 13 is a diagram showing docking of the compound D4 with a PKC protein molecule.

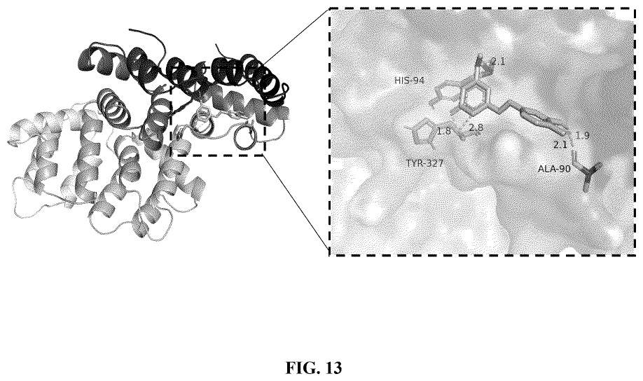

FIG. 14 is a diagram showing docking of the compound D7 with a PKC protein molecule.

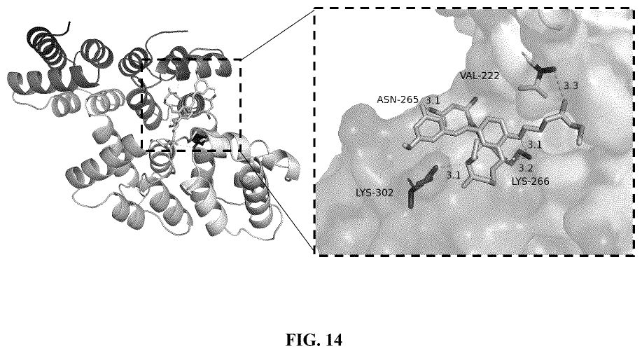

FIG. 15A shows detection of lipid-lowering activity of the compound D4.

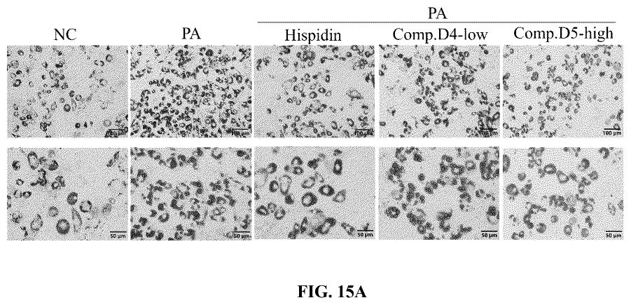

FIG. 15B shows detection of lipid-lowering activity of the compound D4

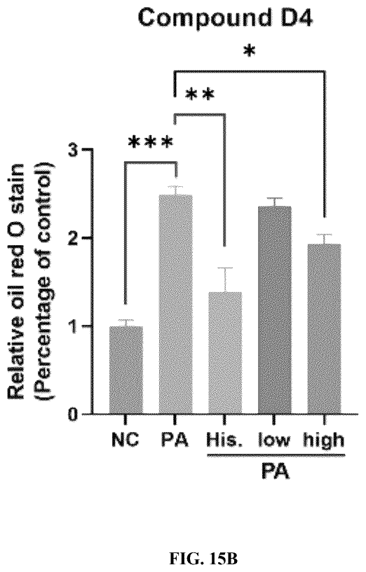

FIG. 16A shows detection of lipid-lowering activity of the compound D7.

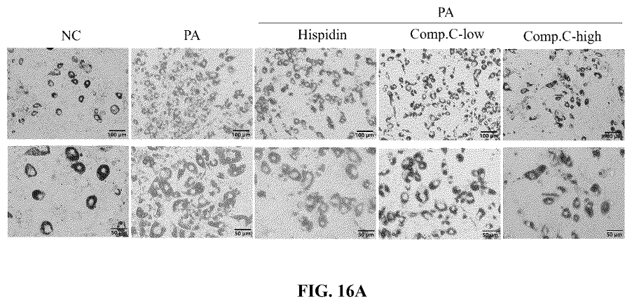

FIG. 16B shows detection of lipid-lowering activity of the compound D7.

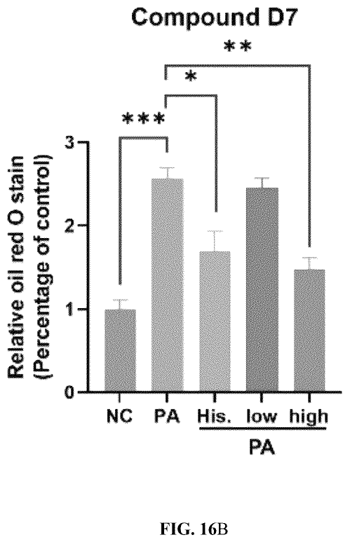

### DETAILED DESCRIPTION OF THE EMBODIMENTS

The technical solutions in the embodiments of the present disclosure will be clearly and completely described hereafter. All other embodiments obtained by a person of ordinary skill in the art without creative efforts on the basis of the embodiments of the present disclosure shall fall within the scope of protection of the present disclosure.

In the present disclosure, firstly phosphogypsum is mixed with a composite solvent, stirred, filtered, reacted at a high temperature, and filtered by suction under vacuum. The present disclosure will now be described in further detail with reference to implementation examples.

### Example 1

A raw material 4-hydroxy-6-methyl-2-pyrone (500 mg, 3.97 mmol) was dissolved with tetrahydrofuran (30 mL), added with anhydrous potassium sulfate (3 g, 17.2 mmol) after the solution was clarified, and stirred, dropwise added with dimethyl sulfate (0.38 mL, 3.97 mmol) (in a molar ratio of 4-hydroxy-6-methyl-2-pyrone:dimethyl sulfate of 1:1), condensed to reflux in an oil bath at 50° C. under magnetic stirring, monitored by TLC (ethyl acetate:petroleum ether=1:2) until the raw material point disappeared, and filtered while hot. The filtrate was retained, spin-dried and then acidified with addition of dilute hydrochloric acid. The resulting solution was subjected to gradient elution by column chromatography in a mixed solvent of petroleum ether and ethyl acetate (PE/EA) to separate the mixture, and the organic layer was dried over anhydrous sodium sulfate and concentrated under reduced pressure to obtain a compound D1.

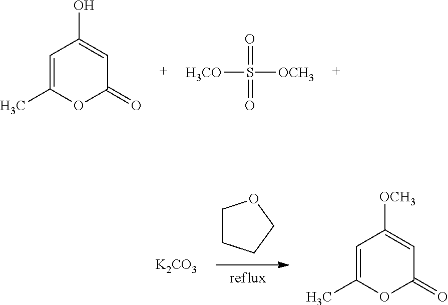

### Example 2

3,4-dihydroxybenzaldehyde (2.0 g, 15 mmol) was dissolved with N,N-dimethylformamide (15 mL), added with anhydrous potassium sulfate, meanwhile continuously dropwise added with dichloromethane (30 mL), condensed to reflux in an oil bath at 100° C. under magnetic stirring, monitored by TLC until the raw material point disappeared, and filtered while hot. The filtrate was washed with an aqueous solution and a 2% sodium chloride solution sequentially. The organic layer was dried over a small amount of anhydrous sodium sulfate, concentrated under reduced pressure, then diluted with ethyl acetate:petroleum ether=3:1 as an eluent, and separated by column chromatography to obtain a compound D2.

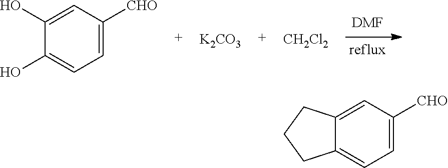

### Example 3

The compound D1 (1.125 g, 8 mmol) and the compound D2 (1.0 g, 6.7 mmol) were dissolved with magnesium methoxide (28 mL, 20 mmol) (in a molar ratio of magnesium methoxide:D1:D2 of 3:1:1), condensed to reflux for reaction under magnetic stirring in an oil bath at 60° C., monitored by TLC until the reaction was complete, and spin-dried to remove the organic solvent and obtain a crude product. The crude product was dissolved with acetic acid, extracted for 2-3 times over a saturated NaHCO₃ and NaCl solutions. The organic layer was retained, dried with addition of a small amount of anhydrous Na₂SO₄ powder, and spin-dried to remove the organic solvent and obtain a yellow solid. The solid was added with dichloromethane and dissolved by ultrasonication or low-temperature reflux, allowed to stand in a refrigerator at 4° C. for crystallization, and subjected to suction filtration under reduced pressure to obtain a compound D3.

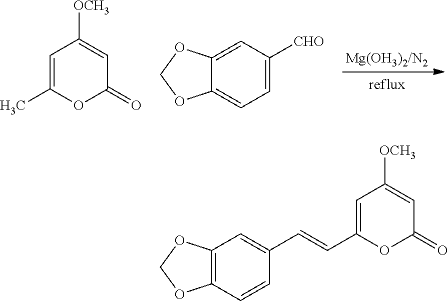

### Example 4

The compound D3 (820 mg, 3.01 mmol) was dissolved with dichloromethane (100 mL), added with boron trichloride (9.03 mL, 9.03 mmol) (in a molar ratio of D3:boron trichloride of 1:3), condensed to reflux under stirring in an oil bath at 40° C. under the protection of nitrogen, monitored by TLC until the reaction was complete, and then spin-dried to remove the organic solvent and obtain a brown solid. The solid was added with a small amount of methanol and dissolved by sonication or low-temperature reflux. The solution was placed into and cooled in a refrigerator at 4° C. for crystallization, and subjected to suction filtration under reduced pressure to obtain a compound D4.

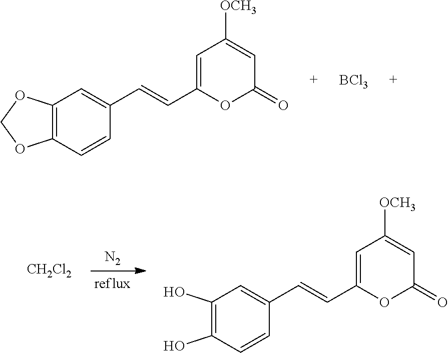

### Example 5

The compound D4 (80 mg, 0.308 mmol) and a small amount of solid potassium carbonate (2 g, 14.5 mmol) were taken, dissolved with DMF, added with reagents a-j (in a molar ratio of D4 to reagents a-j of 1:5) respectively, condensed to reflux in an oil bath at 50° C., and monitored by TLC for a reaction progress, and extracted for multiple times after the reaction was complete. The organic layer was retained and spin-dried sufficiently to remove the organic solvent and obtain a powder product. The powder product was poured into a chromatography column and spread evenly, subjected to gradient elution by column chromatography with a mixed solvent of petroleum ether and ethyl acetate (PE/EA) as the eluent to separate the mixture. The organic layer was dried over anhydrous sodium sulfate and concentrated under reduced pressure to obtain the compound D1. The solution was subjected to gradient elution by column chromatography to separate the mixture, and spin-dried to obtain compounds D5-D14 (corresponding to R₁=R₂=a-j, respectively).

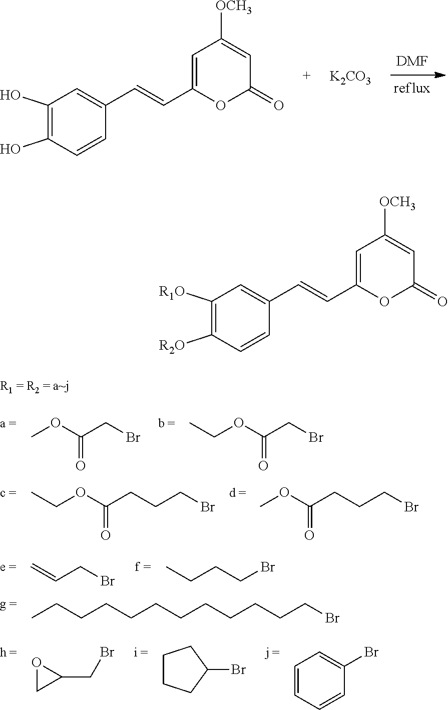

### Example 6

The compounds D4 and D7 were docked with PKC protein target molecules. The compounds D4 and D7 used in this docking were constructed utilizing KingDraw and saved as a file in a sdf format, serving as ligand molecules for molecular docking. Thereafter, the file was imported into Pymol and Autodock software for optimization treatment and exported as a PDBQT file. The PKC (PDBID:5lpx) protein structure was derived from RCSB database (https://www.rcsb.org/). The protein structure was subjected to treatment such was removal of water molecules, removal of ligands, and hydrogenation on a Pymol and Autodock platform. The protein was subjected to energy minimizing and geometric structure optimization, and then exported as a PDBQT file. The treatment and optimization of molecular docking were completed using a Grid module in the Autodock software. The PDBQT file of the PKC protein was imported into the software, so as to allow a box to fully encapsulate the protein, and then perform molecular docking. Additionally, a complex of proteins and small molecules was visualized and analyzed with Pymol.

### Example 7

Method for evaluating in vitro cytotoxicity:

### (1) Cell Lines and Cell Culture

Mouse 3T3-L1 preadipocytes were provided by the Cell Resource Center of the Shanghai Institutes for Biological Sciences, Chinese Academy of Sciences. Cells were cultured in an incubator (Thermo Scientific, USA) under conditions of 5% CO₂ and 37° C., and all cells were cultured under sterile conditions.

### (2) Detection of Effect of Compound (D4) on Lipid Droplets of 3T3-L1 Cells by Oil Red O

A 12-well plate was used. Cells were collected and plated, and 600-700 μL of a culture solution was added per well. The cells were cultured in an incubator with 5% CO₂ and 37° C. until the cell confluence reached approximately 50%, added with an inducer I (containing 1 μM of dexamethasone, 1 μM of rosiglitazone, 10 μM of indomethacin, 0.5 μM of IBMX, and 5 μg/mL of insulin) for induction for 4-6 days. Once dense small lipid droplets of the cells appeared, the inducer was replaced with an inducer II (5 μg/mL of insulin). The medium was replaced every other day. The lipid droplets gradually changed from small and dense to large and round over 8-10 days. Almost all adipocytes changed from elongated spindle shapes to round shapes, indicating successful adipocyte induction. After successful cell induction, the cells were divided into 6 groups: an NC group (normal control group), a PA group (model group), a PA+Hispidin group (positive control group), a PA+compound D4 low-dose group, and a PA+compound D4 high-dose group. The groups were cultured in a cell incubator with 5% CO₂ and 37° C. for 24 h. After the treatment was completed, the medium was discarded, the cells were washed twice with PBS, fixed with a 4% paraformaldehyde fixation solution for 30 min, washed for 3 times with PBS, and added 1 mL of an Oil Red O staining working solution for staining at room temperature with protection from light for 30 min. The staining solution was discarded, the residual staining solution was washed away with PBS, 1 mL of PBS was added per well, and the cell lipid droplets were observed under a microscope and photographed.

The above is based on enlightenment from the ideal embodiments of the present invention. Through the above description, various changes and modifications can be made by relevant personnel within the scope of not deviating from the technical idea of the present disclosure. The technical scope of the present disclosure is not limited to the content of the specification, but must be determined according to the scope of the claims.

What is claimed is:

1. A Hispidin derivative, which is:

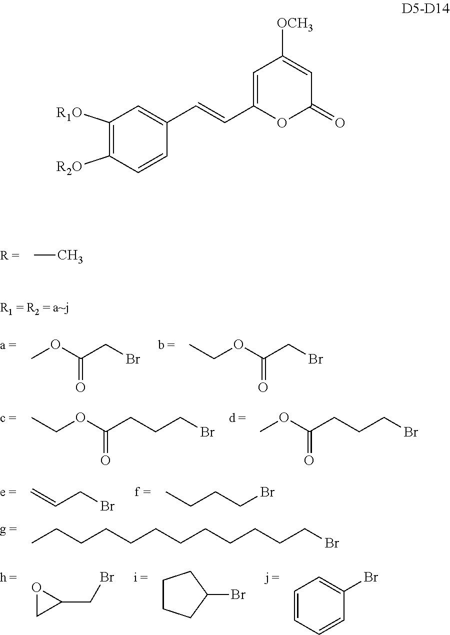

2. A method for preparing the Hispidin derivative according to claim 1, comprising the following steps:

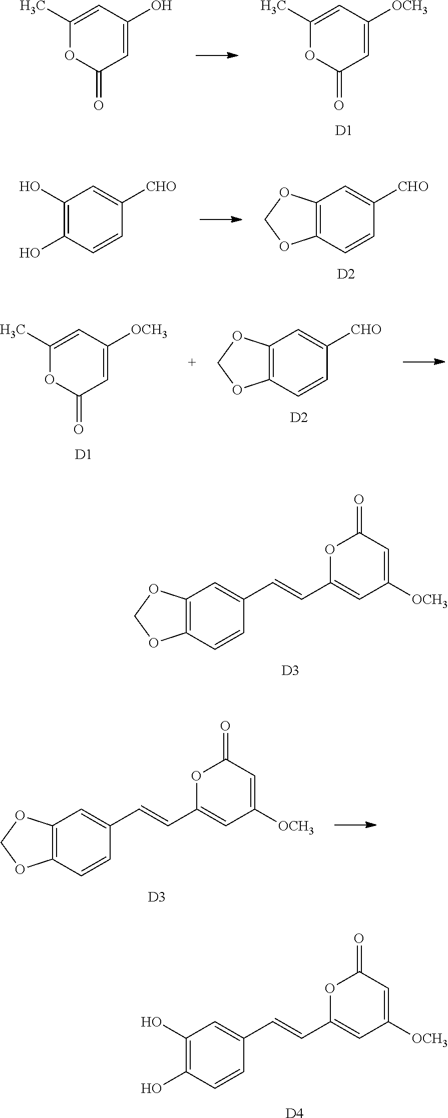

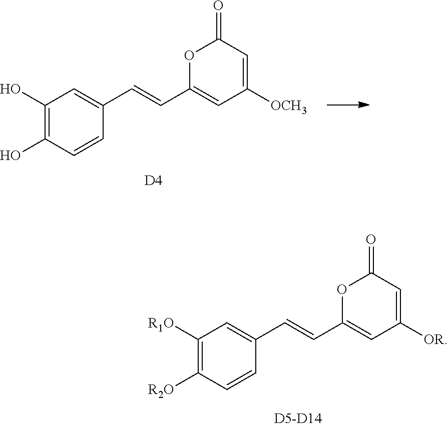

3. A method for preparing the Hispidin derivative according to claim 1, comprising the following steps:
S1: dissolving a raw material 4-hydroxy-6-methyl-2-pyrone with tetrahydrofuran, adding anhydrous potassium sulfate, stirring, then dropwise adding dimethyl sulfate and condensing to reflux, monitoring by TLC until a raw material point disappears, filtering while hot, collecting a filtrate, spin-drying, then acidifying with addition of dilute hydrochloric acid, and sequentially washing, drying and concentrating a resulting solution under reduced pressure to obtain a compound D1;
S2: dissolving 3,4-dihydroxybenzaldehyde with N,N-dimethylformamide, adding anhydrous potassium carbonate, meanwhile dropwise adding dichloromethane, condensing to reflux, monitoring by TLC until a raw material point disappears, filtering while hot, sequentially washing, drying and concentrating a filtrate under reduced pressure, then eluting, and separating by column chromatography to obtain a compound D2;
S3: dissolving the compound D1 and the compound D2 with magnesium methoxide, heating, condensing to reflux for reaction; monitoring by TLC until the reaction is complete, spin-drying to remove an organic solvent and obtain a crude product, dissolving the crude product with acetic acid, then sequentially extracting, drying and spin-drying to obtain a yellow solid, dissolving the solid with addition of dichloromethane under reflux, allowing to stand for crystallization, and performing suction filtration under reduced pressure to obtain a compound D3;
S4: dissolving the compound D3 with dichloromethane, adding boron trichloride and condensing to reflux under the protection of nitrogen, monitoring by TLC until the reaction is complete, then spin-drying to remove the organic solvent and obtain a gray-green solid, dissolving the solid with addition of methanol under reflux, placing and cooling the solution in a refrigerator at 4° C. for crystallization, and filtering under reduced pressure to obtain a gray-green granular compound D4; and
S5: dissolving the compound D4 with N,N-dimethylformamide, adding anhydrous potassium carbonate, heating and condensing to reflux for reaction; monitoring by TLC until the reaction is complete, washing the reaction solution with a saturated sodium chloride solution and water sequentially, drying with anhydrous sodium sulfate, concentrating under reduced pressure, then eluting, and separating by column chromatography to obtain compounds D5-D14.

4. The method for preparing the Hispidin derivative according to claim 2, wherein in the step S1: the raw material 4-hydroxy-6-methyl-2-pyrone (500 mg, 3.97 mmol) is dissolved with tetrahydrofuran (30 mL), added with anhydrous potassium sulfate (3 g, 17.2 mmol) after the solution is clarified, and stirred, dropwise added with dimethyl sulfate (0.38 mL, 3.97 mmol) at an equivalent molar mass as 4-hydroxy-6-methyl-2-pyrone (in a molar ratio of 4-hydroxy-6-methyl-2-pyrone:dimethyl sulfate of 1:1), condensed to reflux in an oil bath at 50° C. under magnetic stirring, monitored by TLC (ethyl acetate:petroleum ether=1:2) until the raw material point disappears, and filtered while hot, the filtrate is retained, spin-dried and then acidified with addition of dilute hydrochloric acid, a resulting solution is subjected to gradient elution by column chromatography in a mixed solvent of petroleum ether and ethyl acetate (PE/EA) to separate the mixture, and an organic layer is dried over anhydrous sodium sulfate and concentrated under reduced pressure to obtain the compound D1.

5. The method for preparing the Hispidin derivative according to claim 2, wherein in the step S2: 3,4-dihydroxybenzaldehyde (2.0 g, 15 mmol) is dissolved with N,N-dimethylformamide (15 mL), added with anhydrous potassium sulfate, meanwhile continuously dropwise added with dichloromethane (30 mL), condensed to reflux in an oil bath at 100° C. under magnetic stirring, monitored by TLC until the raw material point disappears, and filtered while hot, the filtrate is washed with an aqueous solution and a 2% sodium chloride solution sequentially, the organic layer is dried over a small amount of anhydrous sodium sulfate, concentrated under reduced pressure, then diluted with ethyl acetate:petroleum ether=3:1 as an eluent, and separated by column chromatography to obtain the compound D2.

6. The method for preparing the Hispidin derivative according to claim 2, wherein in the step S3: the compound D1 and the compound D2 are dissolved with magnesium methoxide (in a molar ratio of magnesium methoxide:D1:D2 of 3:1:1), heated, condensed to reflux for reaction; monitored by TLC until the reaction is complete, and spin-dried to remove an organic solvent and obtain a crude product, the crude product is dissolved with acetic acid and then sequentially extracted, dried and spin-dried to obtain a yellow solid, the solid is dissolved with addition of dichloromethane under reflux, allowed to stand for crystallization, and subjected to suction filtration under reduced pressure to obtain the compound D3.

7. The method for preparing the Hispidin derivative according to claim 2, wherein in the step S4: the compound D3 (820 mg, 3.01 mmol) is dissolved with dichloromethane (100 mL), added with boron trichloride (9.03 mL, 9.03 mmol) (in a molar ratio of D3:boron trichloride of 1:3), condensed to reflux under stirring in an oil bath at 40° C. under the protection of nitrogen, monitored by TLC until the reaction is complete, and then spin-dried to remove the organic solvent and obtain a brown solid, the solid is added with a small amount of methanol and dissolved by sonication or low-temperature reflux, and the solution is placed into and cooled in a refrigerator at 4° C. for crystallization, and subjected to suction filtration under reduced pressure to obtain the compound D4.

8. The method for preparing the Hispidin derivative according to claim 2, wherein in the step S5: the compound D4 (80 mg, 0.308 mmol) and a small amount of solid potassium carbonate (2 g, 14.5 mmol) are taken, dissolved with DMF, added with reagents a-j (in a molar ratio of D4 to reagents a-j of 1:5) respectively, condensed to reflux in an oil bath at 50° C., and monitored by TLC for a reaction progress, and extracted for multiple times after the reaction is complete, the organic layer is retained and spin-dried sufficiently to remove the organic solvent and obtain a powder product, the powder product is poured into a chromatography column and spread evenly, subjected to gradient elution by column chromatography with a mixed solvent of petroleum ether and ethyl acetate (PE/EA) as the eluent to separate the mixture, and the organic layer is dried over anhydrous sodium sulfate and concentrated under reduced pressure to obtain the compound D1, the solution is subjected to gradient elution by column chromatography to separate the mixture, and spin-dried to obtain the compounds D5-D14 (corresponding to R=—CH₃, R₁=R₂=a-j, respectively).

9. A lipid-lowering drug, comprising the Hispidin derivative according to claim 1, wherein D4 and D7 form sites containing hydrophobic interactions and hydrogen bond interactions by binding to an amino acid at an active site of a PKC protein.
